(19) United States
(12) Patent Application Publication
(10) Pub. No.: US 2016/0369357 A1
(43) Pub. Date: Dec. 22, 2016

(54) METHOD FOR DIAGNOSING, QUANTIFYING, TREATING, MONITORING OR EVALUATING CONDITIONS, DISEASES OR DISORDERS ASSOCIATED WITH HUMAN PAPILLOMA VIRUS (HPV) INFECTION

(71) Applicant: Donald Charles Taylor, Comox, BC (CA)

(72) Inventor: Donald Charles Taylor, Comox, BC (CA)

(21) Appl. No.: 15/189,811

(22) Filed: Jun. 22, 2016

Related U.S. Application Data
(60) Provisional application No. 62/182,951, filed on Jun. 22, 2015.

(51) Int. Cl. C12Q 1/70

(52) U.S. Cl. CPC C12Q 1/708; C12Q 2600/118; C12Q 2600/16; C12Q 2600/158

(57) ABSTRACT

A method of diagnosing, quantifying, treating, monitoring or evaluating a condition in a subject is provided. The method comprises obtaining a sample from the subject, detecting and determining the presence or quantity of one or more than one human papilloma virus (HPV) in the sample, wherein the presence or quantity of the one or more than one HPV is indicative of the presence of the condition in the subject or an increased likelihood of the subject for developing the condition when compared to a control. The condition may comprise autoimmune disorders, chronic neurodegenerative diseases, neurodevelopmental disorder, neoplasm, blood cancers such as lymphoma or leukemia, Chronic Fatigue Syndrome or Fibromyalgia.

### CROSS-REFERENCE TO RELATED APPLICATIONS

This U.S. Non-Provisional Application claims priority to U.S. Provisional Patent Application Ser. No. 62/182,951 filed Jun. 22, 2015, and entitled “Method for Diagnosing, Quantifying, Treating, Monitoring or Evaluating Conditions, Diseases or Disorders Associated with Human Papilloma Virus (HPV) Infection,” which is incorporated herein by reference in its entirety.

### FIELD OF INVENTION

The disclosure relates to a method of diagnosing, quantifying, treating, monitoring or evaluating conditions, diseases or disorders associated with human papilloma virus (HPV) in a subject, by detecting and determining the presence or quantity of one or more HPV in samples from subjects with the condition, diseases or disorders for diagnosing, treating, monitoring or evaluating the condition.

### BACKGROUND OF THE INVENTION

Human papillomaviruses (HPVs) are a group of double-stranded, nonenveloped, small DNA viruses that are widely prevalent among human populations (See FIG. 1). To date, over 180 types of HPV have been isolated and identified from different body sites and have been collected in two HPV databases (See: http://pave.niaid.nih.gov, http://www.hpvcenter.se), and the number is growing (See: Ma et al. J. Virol. May 2014 vol. 88 no, 9 4786-4797).

The HPV genome is double-stranded DNA that codes for eight proteins. These proteins are named for when they are expressed during the virus' occupation of its host: early (E) and late (L). E1, 2, 4, and 5 are important to genome replication. E1 also prevents the virus' genome from integrating into the host's genome, and initiates gene expression when complexed with E2, E6 and E7 are oncogenes, and, unchecked, can cause tumorous growth of the host cell. E2 and E4 normally inhibit this action. The late proteins, L1 and L2, are capsid proteins and are thus only expressed when the virus copies are repackaged to be released from the host cell (See: Doorbar 2005; J Clin. Virol. 32:S7-15).

HPVs are divided into five genera: alpha (also known as A supergroup), beta (also known as B supergroup), gamma, mu, or nu (See FIG. 2). Mu and nu-papillomaviruses are also classified as E supergroup. Supergroup A encompasses all genitally transmitted papillomaviruses, as well as some cutaneous viruses. The genitally transmitted viruses present as mucosal lesions, while the cutaneous variety are the primary cause of skin warts. Viruses in supergroup B also form cutaneous warts similar to those from group A, and they are one cause of non-melanoma skin cancer. Supergroup E is comprised of a third type of virus that causes cutaneous warts, HPV types belonging to different genera have less than 60% similarity, based on the nucleotide sequence of the capsid protein L1. Different viral species within a genus share between 60 and 70% similarity (See: Bzhalava et al. Virology Volume 445, issues 1-2, October 2013, Pages 224-231).

All HPVs develop and reproduce solely in keratinocytes, or keratin-producing epithelial cells, the differentiation of which is critical to the papillomavirus's own development. The virus first infects keratinocyte stem cells, which live in the basal layer of the epithelium, through a breach in the upper layers of the epithelitun. Upon infection, the virus takes advantage of the cell's replication machinery to reproduce its genome several times, so that each infected cell contains a low viral load of about 50 copies. As the cells proliferate, they move towards the outer layers of the epithelium; the viruses proliferate as well, but do not amplify their genome to escape detection by the immune system. When the host keratinocyte reaches S-phase in the differentiation compartment of the epithelium, the papillomavirus replicates its genome to the critical limit of about 1000 copies. In fact, the virus releases growth promoters E6 and E7 at this stage in order to stimulate the host's movement into this phase. When the keratinocyte reaches the superficial epithelium and dies, the genomes are repackaged into capsids and shed from the cell (See: Doorbar 2005; J Virol. 32:S7-15).

Infection with HPV can present a wide variety of clinical manifestations of the skin and mucosa, most commonly as warts, either skin or genital, although many HPV infections, especially those of the skin, are often present without clinical symptoms. The most important HPV-associated disease is undoubtedly cancer. Cervical cancer and many other anogenital cancers are caused by HPV infections, and many other cancer forms have either been proven or are suspected to be linked to HPV.

In the case of immunocompromised patients, the risk of HPV infection was reported to be much greater than the general population due to high-load, persistent infection with oncmenic HPV genotypes. The prevalence of HPV infection was shown to increase in organ transplant recipients, human immunodeficiency virus (HIV)-infected women, and patients with systemic autoimmune diseases such as systemic lupus erythematosus (SLE). Furthermore, it has been found that patients with active SLE had greater prevalence of high-risk HPV infection and of abnormal cervical cytology compared with controls, and that SLE itself was identified with independent risk factors for high risk HPV infection among Korean women, and Pap smear abnormalities (See: Lee et al 2010 (J Korean Med Sci. 2010 October; 25(10); 1431-1437).

Infection with HPV has also been linked with an increased risk of cardiovascular disease (See: Kuo H K, Fujise Journal of the American College of Cardiology 58 (19): 2001-6.). Furthermore, it has been shown that there is an association between human papillomavirus DNA and temporal arteritis (See: Mohammadi et al. (BMC Musculoskeletal Disorders 2012, 13:132).

It has been reported that human papillomavirus 16 (HPV16) can be linked to a common form of childhood epilepsy (See: Chen et al. 2012 (Ann Neurol. 2012 December; 72(6):881-92). It was shown that HPV16 may be present in the human brain, and they found that when they added a viral protein to the brains of fetal mice, the mice all demonstrated the same developmental problems in the cerebral cortex associated with this type of epilepsy, called focal cortical dysplasia type IIB (FCDIIB). The findings suggest that HPV could play a role in the development of epilepsy.

Studies have reported HPV findings in cancers that occur at sites where direct HPV infection is usually not seen, including colorectal, and lung and breast cancer. Detection of HPV in cancers like this raises the question how HPV got to the site of cancer. It has long been believed that papillomavirus cannot be spread to different body sites through blood, as papillomaviruses do not give rise to viremia. One possibility is that it was transported in blood, and HPV DNA has indeed been reported to be found in circulating blood, including peripheral blood mononuclear cells (PBMCs), sera, plasma, and arterial cord blood (See: Dong et al., 2002, Cancer Epidemiol Biomarkers Prev 11:3-6., 9). It has also been reported that 8.3% of healthy male blood donors in their study were positive for HPV (See: Chen et al., 2009, Med Virol. 2009 Oct; 81(10):1792-61. Chen et al. isolated a wide variety of different HPV types from PBMCs, belonging to the cutaneous beta and gamma papillomavirus genera and mucosal alpha papillomaviruses. High-risk HPV types that are linked to cancer development were detected in 1.7% of the PBMCs.

All papillomaviruses, including HPV, are obligatory intranuclear organisms with specific tropism for keratinocytes and lymphatic cells. Three possible courses of events can follow upon HPV entry into cells: (1) viral DNA are maintained as intranuclear, extrachromosomal, circular DNA episomes, which replicates synchronously with the host cell, establishing a latent infection; this period usually last an average of 1 to 8 months; although, in some patients this latency may last for many years or decades (2) conversion from latent into productive infection with assembly of complete infective virions; and, (3) integration of viral DNA into host cellular genome, a phenomenon seen in HPV infections associated with malignant transformation.

Like other viruses, HPV has the ability to establish a chronic, dormant (latent) infection and the ubiquity of latent papillomavirus infections is emphasized by the frequent, often acute outbreak of warts in immunosuppressed patients and pregnant women. In latency, viral DNA replication and transcription are maintained at very low levels and regulated by negative cellular factors (e.g., NF-IL6, p53, Oct-1, YY1) and low levels of early viral proteins (E1 and E2). For example, the viral E1 replication protein functions as an E2 co-repressor when bound to the origin of DNA replication. Persistent HPV infections are associated with a number of skin and cervical cancers, but could be associated with other conditions

Many viruses establish persistent infections that are characterized by continuous low or high levels of viral replication example human immunodeficiency virus and hepatitis B virus) or by periodic reactivation of a latent infection following apparently disease-free intervals (for example herpes simplex virus) (See: Tyler, K. and Nathanson, N. in Fields Virology 4th edn (eds Fields B N, Knipe, D. M., and Howley, P. M.), 220-222 (Lippincott Williams & Wilkins, Philadelphia, 2001) (FIG. 3). As illustrated therein, acute infection is associated with clinical symptoms and the release of an infectious virus, (e.g. influenza virus). Persistent infection is associated with the production of an infectious virus, HBV for example, for the lifetime of the host. Latent infection as seen in herpes simplex virus (HSV) infection is a variation of persistent infection in which the acute infection is followed by a quiescent phase in which the virus productive cycle is absent or significantly reduced. The viral genome remains in a ‘silent’ state but can be intermittently reactivated into bouts of productive infection. Slow virus infection, as seen with HIV is yet another version of persistent infection typified by long periods (years) between primary infection and the development of fatal symptoms: production of an infectious virus is either continuous at a low level or absent until failing immune control results in overwhelming virus production.

A number of viruses are capable of invading and establishing latent infections in nervous tissue. Such viruses have been shown to produce slow, chronic, or progressive nervous system diseases (See: Boldogh et al. Medical Microbiology, 4th edition. Chapter 46). Many chronic, degenerative nervous system diseases are related to viral persistence. Persistence in the nervous system probably involves some unique mechanisms that take advantage of the many types of specialized cells and the immunologically privileged status of the central nervous system. For example during acute HSV infection, virus and/or viral components (e.g., nucleocapsids) containing viral genetic material ascend in nerve axons from the initial site of infection to the sensory ganglia mainly the trigeminal ganglia HSV-1, and the lumbar and sacral ganglia for HSV-2. In the sensory ganglia, the virus may cause a cytolytic infection or establish a latent, noncytolytic infection. Sympathetic ganglia and other cell types of the central nervous system may also serve as sites of virus latency.

In the case of Varicella-Zoster Virus infection, after recovery from acute varicella (chickenpox), the virus establishes latency in multiple ganglia of the human neuraxis. Years later, the virus may reactivate, and the distribution of lesions in the skin corresponds closely to areas of innervation (dermatome) from an individual dorsal root ganglion. In immunocompromised patients, life-threatening disseminated infections can occur.

Measles is normally an acute self-limited disease in which the virus appears to be eliminated. In some individuals, however, the virus persists in the brain despite apparent humoral and cellular immune responses. Possible mechanisms of persistence include the immunologically privileged status of the brain, antiviral antibody-induced internalization of viral antigens, altered and restricted virus expression and replication as a result of mutations in the virus genome.

Furthermore, viral infection has also been linked to neurodegenerative and neurobehavioral diseases (See: Nicolson, Lab Med. 2008; 39(5):291-299). For example HSV infections have been found in Alzheimer's disease (AD), and an interesting relationship has developed between the presence of HSV in AD. It had been noted in HSV but not a related neurotrophic virus, varicella zoster virus, was found often in AD brains and may be linked to patients who have the AD risk factor ApoE e4 allele.

### SUMMARY OF THE INVENTION

The present disclosure relates to a method of diagnosing, quantifying, treating, monitoring or evaluating a condition in a subject comprising determining the presence or quantity of one or more than one human papilloma virus (HPV) in a sample from the subject, wherein the presence or quantity of the one or more than one HPV is indicative of the presence of the condition in the subject or indicative of a predisposition to the condition when compared to a control.

The quantity of the one or more than one HPV may comprise the viral load of one or more than one HPV in a sample from the subject. The condition may comprise an autoimmune disorder, a chronic neurodegenerative disease, a neurodevelopmental disorder, neoplasm, blood cancers such as lymphoma or leukemia, Chronic Fatigue Syndrome or Fibromyalgia. The autoimmune disorder may be Systemic lupus erythematosus (SLE) or Multiple Sclerosis (MS). The chronic neurodegenerative disease may be Dementia such as for example, Alzheimer's disease, Parkinson's disease or Amyotrophic lateral sclerosis (ALS), and the neurodevelopmental disorder may be Autism or autism spectrum disorders such as Asperger syndrome. The method may further comprise treating the subject for one or more than one HPV infection or may comprise vaccinating the subject against one or more than one HPV. The sample from the subject may be blood, plasma, peripheral blood mononuclear cells (PBMC), or other blood compartment or fluid such as cerebrospinal fluid or saliva.

The viral load may be determined by quantifying the genome numbers of the one or more than one HPV and the HPV genome numbers in the sample may be greater than the genome numbers in a sample of the control. The genome numbers may be determined by quantitative polymerase chain reaction (PCR). The genome numbers may be greater than 4 copies per μl as measured by HPV-DNA quantitative PCR using universal primer SPF10. The viral load may be above a threshold amount; the threshold amount being indicative of the presence of the condition in the subject or an increased likelihood of the subject developing the condition when compared to a control subject. The threshold amount may be a copy number greater than 4 copies per as measured by HPV-DNA quantitative PCR using universal primer SPF10.

The one or more than one HPV may be one or more beta, gamma or alpha HPV or a combination thereof. Furthermore, the one or more HPV may comprise one or more high risk HPV type, one or more low risk HPV type HPV or a combination thereof. The HPV may comprise one or more HPV type 1, 2, 4, 6, 11, 16, 18, 26, 30, 31, 33, 34, 35, 39, 40, 41, 42, 43, 44, 45, 51, 52, 54, 55, 56, 57, 58, 59, 61, 62, 64, 67, 68, 69, 70 or a combination thereof.

Furthermore, the present disclosure also relates to a method of comparing the incidence of a HPV infection among more than one population of subjects with a condition, the method comprising

a) determining the viral load of one or more HPV in a representative number of subjects of each population;

b) calculating the percentage of subjects in each population having a viral load above a threshold; and

c) correlating the percentage of subjects in each population to the relative incidence of HPV infection. The condition may comprise an autoimmune disorder, a chronic neurodegenerative disease, a neurodevelopmental disorder, neoplasm, blood cancers such as lymphoma or leukemia, Chronic Fatigue Syndrome or Fibromyalgia. The autoimmune disorder may be Systemic lupus erythematosus (SLE) or Multiple Sclerosis (MS). The chronic neurodegenerative disease may be Alzheimer's disease, Parkinson's disease or Amyotrophic lateral sclerosis (ALS) and the neurodevelopmental disorder may be Autism or autism spectrum disorders such as Asperger syndrome.

The disclosure further relates to a method for predicting the development of a condition in a human subject, comprising contacting a biological sample of the human subject with a diagnostic reagent that can detect one or more than one HPV or an antigen thereof, or a humoral or cell-mediated response to HPV or an antigen thereof, in the biological sample,

wherein the presence of one or more than one HPV or an antigen thereof or a humoral or cell-mediated response to HPV or an antigen thereof is associated with an increased risk of developing the condition, wherein the condition comprises an autoimmune disorder, a chronic neurodegenerative disease, a neurodevelopmental disorder, Chronic Fatigue Syndrome or Fibromyalgia.

The disclosure also relates to a method of treating an condition related to HPV comprising:
administering a therapeutically effective amount of an anti-retroviral agent to a to a subject in need thereof, wherein the condition comprises an autoimmune disorder, a chronic neurodegenerative disease, a neurodevelopmental disorder, neoplasm, blood cancers such as lymphoma or leukemia, Chronic Fatigue Syndrome or Fibromyalgia.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1a shows a schematic drawing of a Papilloma Virion. A single molecule of circular dsDNA is contained within the icosahedral capid, which is composed of 72 pentamers of the capsid protein L1.

FIG. 1b shows a transmission electron microscopy (TEM) image of a human papillomavirus.

FIG. 2 shows a phylogenetic tree and the placement of the HPV types found in different organs (See: Ma et al. J. Virol. 2014; 88:4786-4797).

FIG. 3 shows the general pattern of infection for different viruses. To illustrate different patterns of persistent infection, the replication of herpes simplex virus (HSV), hepatitis B virus (HBV) and human immunodeficiency virus (HIV) is plotted as a function of time after infection.

FIG. 4 shows the schematic representation of the locations of the different universal primer sets (MY09/11, GP5+/6+ and SPF 10) on the HPV genome. The circular HPV DNA genome is represented by a single line, and boxes show the positions of the various early (E) and late (L) genes. Within the L1 region, the positions of the amplification targets as well as the expected amplimer sizes for each of the primer sets are indicated (adapted from Meter et al. J Clin Microbiol. 1999 August; 37(8): 2508-2517).

FIG. 5 shows the standard curve for HPV assay using primer pair GP5+/GP6+ generated as a plot of the threshold cycle (y axis) against quantity of standard (log scale). The correlation coefficient R² is 0.978.

FIG. 6 shows the standard curve for HPV assay using primer pair SPF 10 generated as a plot of the threshold cycle (y axis) against quantity of standard (log scale). The correlation coefficient R² is 0.9905.

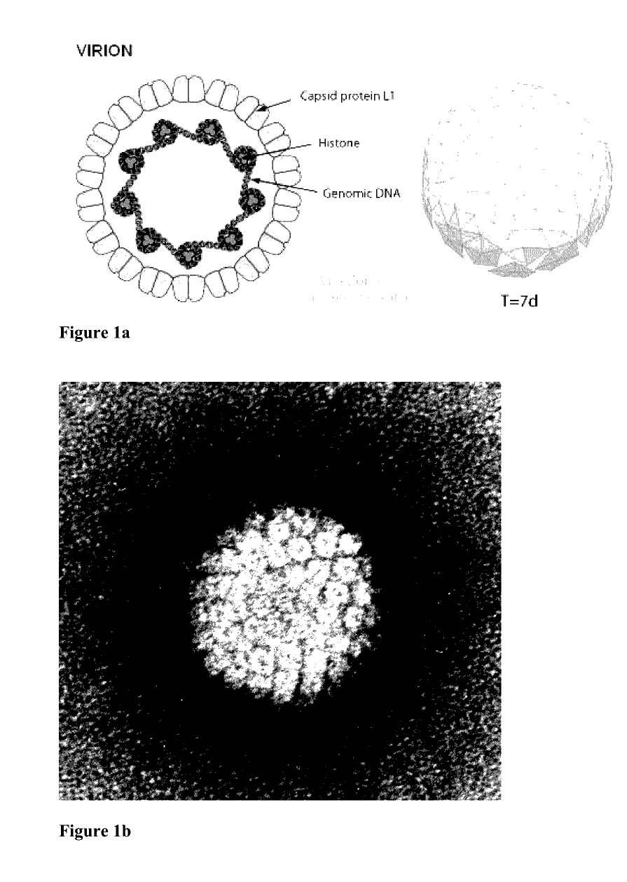

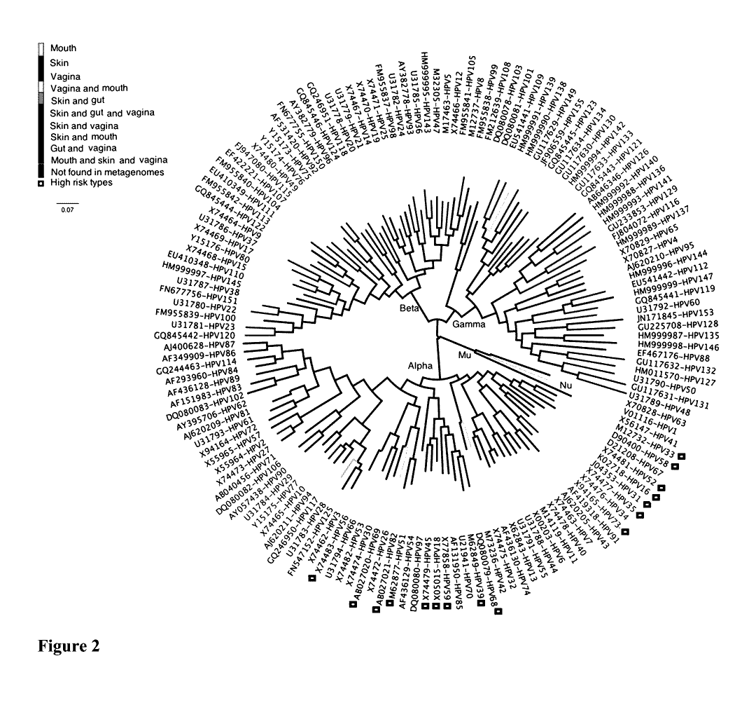

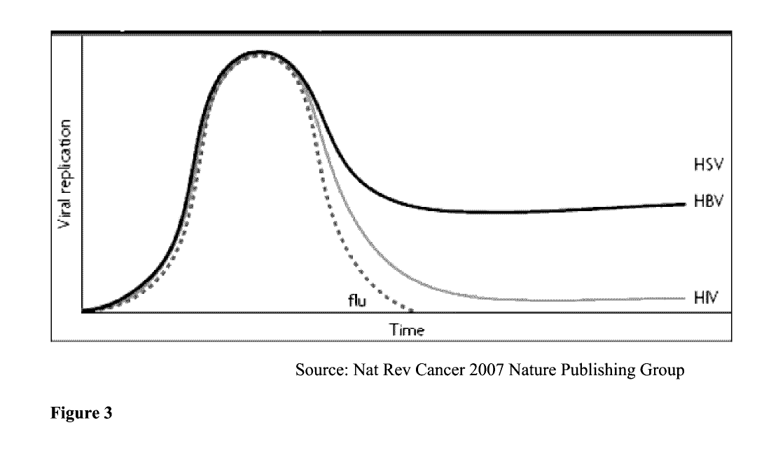

### DETAILED DESCRIPTION

The current disclosure relates to a method diagnosing, treating, monitoring or evaluating a condition in a subject comprising determine the presence or quantity of one or more than one human papilloma virus (HPV) in a sample from the subject, wherein the presence or quantity of the one or more than one HPV is indicative of the presence of the condition in the subject or indicative of a predisposition to the condition when compared to a control, the condition comprising an autoimmune disorder, a chronic neurodegenerative disease, a neurodevelopmental disorder, neoplasm, blood cancers such as lymphoma or leukemia, Chronic Fatigue Syndrome or Fibromyalgia.

Furthermore the disclosure relates to diagnosing, treating, monitoring or evaluating a condition in a subject comprising obtaining a sample from the subject and detecting and determining the viral load of one or more than one HPV in the sample, wherein the viral load of the one or more than one HPV is indicative of the presence of the condition in the subject or an increased likelihood of the subject for developing the condition when compared to a control.

The condition may be an autoimmune disorder such for example Systemic lupus erythematosus (SLE) or Multiple Sclerosis (MS). The condition may further be a Chronic neurodegenerative disease such as for example Alzheimer's disease, Parkinson's disease or Amyotrophic lateral sclerosis (ALS) The condition may further be Chronic Fatigue Syndrome or Fibromyalgia. The condition may also be a neurodevelopmental disorder such as for example Autism or autism spectrum disorders such as Asperger syndrome.

Without wishing to be bound by any particular theory, it is believed that presence of one or more than one HPV and a higher viral load of one or more than one HPV may be associated with a range of conditions for example autoimmune disorders such for example SLE or MS, chronic neurodegenerative diseases such for example Alzheimer's disease, Parkinson's disease or ALS, or a neurodevelopmental disorder such as for example Autism or autism spectrum disorders such as Asperger syndrome and Chronic Fatigue Syndrome or Fibromyalgia.

The presence of one or more than HPV in a sample may determined by methods know within the art for example by PCR to detect HPV DNA or HPV RNA or by immunoassay. For example the sample may be serum or plasma derived from a venous or other blood sample (including a whole blood sample). However, cervical secretions, cervical tissue, tissue from other body parts, or other bodily fluids are known to contain antibodies and may be used as a source of the sample. Once the peptide antigen and sample antibody are permitted to react in a suitable medium, an assay is then performed to determine the presence of an antibody-peptide reaction. Sample collection and immunoassay preparation techniques, as well as other diagnostic methods, are known to those of skill in the art and are not a limitation of the invention. For example, see U.S. Pat. No. 7,267,961 for a method of detection of HPV16 E7 antibodies. In another embodiment, the HPV E6, E7 mRNA assay and methods of U.S. Pat. No. 7,888,032 are utilized.

Furthermore, the presence of HPV may be determined by detecting papillomavirus RNA in a sample for example, a fraction of a bodily fluid by purifying extracellular RNA from the fraction of the bodily fluid to prepare extracted RNA and converting the RNA to cDNA. The cDNA or portion of the cDNA may be used to detect and/or quantify HPV in a sample as is known in the art.

Without wishing to be bound by any particular theory, it is believed that these conditions in a subject are associated with an HPV infection in the subject and more specifically with the viral load of one or more than one HPV in the subject, for example the HPV viral load in blood, plasma or peripheral blood mononuclear cells (PBMC).

The viral load of one or more than one HPV may therefore be determined in a subject with the condition. If the viral load is above a threshold amount the threshold amount being indicative of or associated with the presence of the condition in the subject or an increased likelihood of the subject developing the condition when compared to a control, the subject might be further monitored, treated or assessed for risk of development or progression of the condition. During the treatment of the subject the viral load may be further be determined to assess treatment outcome in relation to symptoms of the condition.

The term “viral load” as used herein refers to the amount of viral particles or fragments thereof in a sample from a subject such as for example biological fluid, such as blood or plasma. Viral load encompasses all viral particles, infectious, replicative and non-infective, fragments thereof. Therefore, viral load represents the total number of viral particles and/or fragments thereof circulating in the biological fluid. Viral load can therefore be a measure of any of a variety of indicators of the presence of a virus, such as viral copy number per unit of blood or plasma, units of viral proteins or fragments thereof per unit of blood or plasma, HPV DNA or RNA copies per milliliter of blood or plasma, or peripheral blood mononuclear cell (PBMC). DNA or RNA copies can be measured using techniques well known in the art, for example, using quantative PCR. If cases of multiple HPV infections are included, use of a composite viral load measurement is the state of the art, meaning that the amount of HPV genomes is summed up for analysis, irrespective of the contribution of the individual genotypes to this sum.

The viral load may for example be determined by amplification of HPV DNA, wherein DNA extracted from clinical samples may be amplified using for example, a set of universal primers, which are directed to highly conserved regions of for example, the HPV L1 gene and are potentially capable of detecting all HPV types. Universal HPV primers are known in the art. The amplification and quantification may further be performed using primers or probes specific to the papillomavirus types and subtypes of interest As discussed above, detection of HPV can be achieved using either broad specificity primers that detect a large number of HPV types (either because they have degenerate positions or because they are to conserved regions of the genome, such as for example, primer sets FAP, SPF10, SPF10+, MY09-MY11, GP5+-GP6+, and PGMY09-PGMY11, as disclosed in Gravitt et al., 2000 J. Clin. Microbiol. 38: 357-361 or by amplifying each type of HPV specifically, or in multiplexed PCR (See for example Francisco Romero-Pastrana International Scholarly Research Network ISRN Microbiology Volume 2012, Article ID 186915, which is herein incorporated by reference). The viral load may be determined using HPV type specific primers for amplification and quantification. For example, the viral load may be determined by using type-specific primer for HPV type 1, 2, 4, 6, 11, 16, 18, 26, 30, 31, 33, 34, 35, 39, 40, 41, 42, 43, 44, 45, 51, 52, 54, 55, 56, 57, 58, 59, 61, 62, 64, 67, 68, 69, 70 or a combination thereof.

Other known methods of HPV detection may also be performed. For example HMI might be detected and amplified by ligase chain reaction, transcription-based amplification system, or PCR using low-temperature ready-to-use moderately thermostable DNA polymerase system.

It has been shown that quantitative PCR assay permits evaluation of viral load in the bloodstream, by determining HPV genome copy numbers. (See: Dong et al. Cancer Epidemiol Biomarkers Prev January 2002 11; 3 which is herein incorporated by reference). Therefore the viral load may for example be determined by quantifying the genome numbers of the one or more than one HPV in a sample. As used herein, the term “quantifying the genome numbers” relates to determining or measuring the number of HPV genomes present in a sample; quantification preferably is absolute, i.e., relating to a specific number of genome molecules, or, more preferably, relative, i.e., measured in arbitrary units. Methods allowing for absolute or relative quantification of HPV genomes are well known in the art. E.g., quantitative PCR methods are methods for relative quantification; if a calibration curve is incorporated in such an assay, the relative quantification can be used to obtain an absolute quantification. Other methods known are, e.g. nucleic acid sequence-based amplification (NASBA) or the Branched DNA Signal Amplification Assay method in combination with dot blot or Luminex detection of amplified polynucleotides. It is to be understood that quantifying genome numbers also relates to a semiquantitative quantification, wherein samples are assigned to one of at least two, at least three, at least four, or at least five categories of genome quantity.

The genome numbers may be quantified by normalizing the amplification products obtained from the genomes, i.e., the genome derived amplification products are art into relation to at least one reference amplification product, thereby, preferably, setting the genome numbers into relation to the number of cells in the sample and/or the efficiency of polynucleotide amplification. Thus the reference amplification product may be a product obtained from a polynucleotide known to have a constant abundancy in each cell, i.e., a polynucleotide comprised in most cells of a sample in approximately the same amount. For example the reference amplification product is amplified from a chromosomal or mitochondrial gene or from the mRNA of a housekeeping gene. In a non-limiting example the reference amplification product is amplified from the beta-globin DNA.

Furthermore, plasmid comprising an HPV genome or portion thereof may be used as a standard or reference for estimating the HPV load in a sample. By way of example and without limitation, the standard may be a plasmid comprising the genome of HPV 16 HPV 18, HPV 31, HPV 33, HPV 35, HPV 39, HPV 45, HPV 51, HPV 52, HPV 56, HPV 58, HPV 59, of HPV 68, or a combination thereof. In one example the standard may comprise the genome of HPV 16.

The genome numbers may furthermore be normalized by setting the normalized genome derived amplification products as defined supra for each HPV genotype into relation to the maximum amount of amplification products obtainable for the HPV genotype. The maximum amount of amplification products may be obtained in a corresponding separate reference amplification reaction using the same oligonucleotide primers and PCR conditions except that a suitable positive control polynucleotide is used in an amount high enough to ensure the synthesis of the maximum amount of amplification products. The skilled artisan knows how to select the positive control polynucleotide and a suitable amount thereof; an example can be found in the accompanying Examples.

The viral load may further be determined by performing real-time PCR analysis (e.g., TaqMan®) on reverse-transcribed RNA isolated from the sample to measure the expression levels of one or more than on HPV. Such methods typically utilize pairs of oligonucleotide primers that are amplify reverse-transcribed HPV RNA. Methods for designing oligonucleotide primers specific for a known sequence are well known in the art. Real-time PCR permits the detection of PCR products at earlier stages of the amplification reaction. Specifically, in real-time PCR the quantitation of PCR products relies on the few cycles where the amount of nucleic acid material amplifies logarithmically until a plateau is reached. During the exponential phase, the amount of target nucleic acid material should be doubling every cycle, and there is no bias due to limiting reagents. Methods and instrumentation for performing real-time PCR are well known in the art. See, for example, Bustin (2000) J. Molec. Endocrinol. 25:169-193; Freeman et al. (1999) Biotechniques 112:124-125; Halford (1999) Nat. Biotechnol. 17:835; and Heid et al. (1996) Genome Res. 6(10):986-994, all of which are herein incorporated by reference in their entirety. Many different dyes and probes are available for monitoring PCR and detecting PCR products, more particularly real-time PCR products. In particular, a 5′ nuclease assay may be used to monitor PCR, particularly real-time PCR (e.g., TaqMan®), and to detect PCR amplification products of HPV RNA. In such 5′ nuclease assays, a fluorogenic/quencher oligonucleotide probe (e.g., a TaqMan® probe) may be added to the PCR reagent mix. The fluorogenic/quencher oligonucleotide probe (e.g., TaqMan® probe) comprises a high-energy fluorescent reporter dye at the 5′ end (e.g., FAM) and a low-energy quencher dye at the 3′ end (e.g., TAMRA or a non-fluorescent quencher). When the fluorogenic/quencher oligonucleotide probe (e.g., TaqMan® probe) is intact, the reporter dye's fluorescent emission is suppressed by the close proximity of the quencher. The fluorogenic/quencher oligonucleotide probe (e.g., TaqMan® probe) may be further designed to anneal to a specific sequence of the HPV between forward and reverse primers, and, therefore, the probe binds to the HPV nucleic acid material in the path of the polymerase. PCR amplification results in cleavage and release of the reporter dye from the quencher-containing probe by the nuclease activity of the polymerase. Thus, the fluorescence signal generated from the released reporter dye is proportional to the amount of the PCR product. Methods and instrumentation (e.g., ABI Prism 7700 Detector; Perkin Elmer/Applied Biosytems Division) for performing real-time PCR using a variety of probes are well known in the art. Moreover, methods for designing appropriate probes for real-time PCR are generally known in the art and commercially available.

Sequences of the HPV genome and HPV genes encoding HPV proteins from a variety of HPV types are known in the art as are the sequences of the transcripts, from which appropriate primers and probes for quantitative or real-time PCR can be designed (see above). For example the complete genome of HPV 16, HPV18, HPV31, HPV33, and HPV45 is provided in Genbank Accession Nos:K02718, X05015, EF4221.23, EF422127, and EF2021.67, respectively. Furthermore, sequences may be obtained from databases for example ‘PaVE: the papillomavirus knowledge source’ (http://pave.niaid.nih.gov) and International Human Papillomavirus Reference Center' (http://www.hpvcenter.se).

The term “comparing” as used herein encompasses comparing the genome number determined in the methods of the present disclosure to a suitable reference amount or control. It is to be understood that comparing as used herein refers to a comparison of amounts. The comparison may be carried out manually or computer-assisted. For a computer-assisted comparison, the value of the determined amount may be compared to values corresponding to suitable references which are stored in a database by a computer program. The computer program may further evaluate the result of the comparison, i.e., automatically provide the desired assessment in a suitable output format.

It is also to be understood that the value of the reference, control or threshold may vary depending on the nature of the target gene and depending on how the amount of a gene product is determined in the sample. For example, if the determination of the amount of the gene product includes amplification of the gene product by PCR, the determined amount of a gene product may depend, e.g., on the oligonucleotides used for the PCR reaction since the amplification efficiency of various oligonucleotide pairs for the amplification of a specific gene product varies. However, the person skilled in the art considers this when calculating the reference amount. Particularly, the person skilled knows that, preferably, the same means and methods have to be used for determining the amounts of a specific gene product in a reference sample and in a test sample.

A genome number in the test sample higher than the reference or control indicates the presence of the condition in the subject or is indicative of a predisposition of the subject to the condition. For example, a genome number in the test sample that is statistically significantly higher than the reference or control indicates the presence of the condition in the subject or indicates a predisposition of the subject to the condition. Whether an amount is statistically significantly higher can be determined by the person skilled in the art using various well known statistic evaluation tools.

As will be understood by those skilled in the art, the aforementioned diagnosing, monitoring or evaluating is usually not intended to be correct for 100% of the subjects to be analyzed. The term, however, requires that the assessment will be valid for a statistically significant portion of the subjects to be analyzed. Whether a portion is statistically significant can be determined by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983, Preferred confidence intervals are at least 90%, at least 95%, at least 97%, at least 98% or at least 99%. The p-values are, preferably, 0.1, 0.05, 0.01, 0.005, or 0.001. Preferably, the probability envisaged by the present disclosure allows that the differentiation will be correct for at least 60%, at least 70%, at least 80%, at least 85%, at least 90%, at least 95% or at least 97% of the subjects of a given cohort.

The HPV genome numbers may for example be greater than 1 copy per sample as measured by HPV-DNA quantitative PCR. Furthermore the HPV genome numbers may for example be greater than 1, 2, 3, 4, 5, 10, 15, 20, 25, 30, 35, 40, 45, 50, 60, 70, 80, 90, 100, 150, 200, 250, 300, 400, 500, 600, 700, 800, 900, 1000, 2000, 3000, 4000, 5000, 7000, 8000, 9000, 10000, 20000, 30000, 40000, 50000, 60000, 70000 or 80000 copies per sample as measured by HPV-DNA quantitative PCR.

The HPV genome numbers may for example be greater than 1 copy per reaction as measured by HPV-DNA quantitative PCR, wherein the reaction volume is for example 10 μl. Furthermore the HPV genome numbers may for example be greater than 1, 2, 3, 4, 5, 10, 15, 20, 25, 30, 35, 40, 45, 50, 60, 70, 80, 90, 100, 150, 200, 250, 300, 400, 500, 600, 700, 800, 900, 1000, 2000, 3000, 4000, 5000, 6000, 7000, 8000, 9000, 10000, 20000, 30000, 40000, 50000, 60000, 70000 or 80000 copies per reaction as measured by HPV-DNA quantitative PCR.

The HPV genome numbers may for example be greater than 1 copy per μl of reaction as measured by HPV-DNA quantitative PCR. Furthermore the HPV genome numbers may for example be greater than 1, 2, 3, 4, 5, 10, 15, 20, 25, 30, 35, 40, 45, 50, 60, 70, 80, 90, 100, 150, 200, 250, 300, 400, 500, 600, 700, 800, 900, 1000, 2000, 3000, 4000, 5000, 6000, 7000, 8000, 9000, 10000, 20000, 30000, 40000, 50000, 60000, 70000 or 80000 copies per μl as measured by HPV-DNA quantitative PCR.

The method of the present disclosure is for example, an in vitro method. Moreover, it may comprise steps in addition to those explicitly mentioned above. For example, further steps may relate to sample pre-treatments or evaluation of the results obtained by the method. The method of the present disclosure is used for diagnosing, monitoring or evaluating a condition in a subject. The method of the present disclosure may also be used for monitoring, confirmation, and sub-classification of the subject. The method may be carried out manually or assisted by automation.

The term “diagnosing” as used herein refers to assessing the probability according to which a subject is suffering or will suffer from a disease or condition referred to in this specification. As will be understood by those skilled in the art, such an assessment is usually not intended to be correct for 100% of the subjects to be diagnosed. The term, however, requires that a statistically significant portion of subjects can be correctly diagnosed to suffer from the disease or condition. Whether a portion is statistically significant can be determined by the methods described herein above.

The term “human papillomavirus” or “HPV” relates to human-infecting members of the family of non-enveloped, dsDNA viruses generally known as Papillomaviridae. HPV which may be divided into genera. Therefore in one embodiment the one or more than one HPV may be one or more than one HPV belonging to one or more than one alpha HPV, one or more than one beta HPV, one or more than one gamma HPV, one or more than one mu HPV, one or more than one nu HPV, or a combination thereof. In one embodiment the one or more than one HPV may be one or more than on beta HPV, one or more than one gamma HPV, one or more than one alpha HPV or a combination thereof. HPV types have also been divided into high and low-risk categories. The one or more than one HPV may therefore comprise one or more than one high risk HPV, one or more than one low risk HPV or a combination thereof. The term high-risk (FIR)-HPV relates to genotypes infecting the human mucosa and associated with cancer. Non limiting examples of HPV are genotypes infecting the human mucosa. The term HR-HPV may include possibly high-risk genotypes, for example, but not limiting HPV 26, 53, 67, 70, 73 and 82 and high-risk HPV genotypes. More specifically, HR-HPV may be for example a high-risk genotypes and may include for example but not limiting HPV 16, 18, 26, 31, 33, 35, 39, 45, 51, 52, 53, 56, 58, 59, 66, 67, 68, 70, 73 and 82. The term low-risk (LR)-HPV relates to HPV genotypes that are not associated with an increased risk of cancer. Low-risk HPV types may include for example but not limiting HPV 2, 3, 6, 11, 13, 32, 40, 42, 43, 44 or 57. In one embodiment the HPV may be HPV 16, including genetic variants of HPV16, for example HPV 16 belonging to a HPV 16 sublineage (see for example Cornet et al. 2012 J Virol 86: 6855-6861, doi: 10.1128/jvi.00483-12 herein incorporated by reference).

The term “sample” refers to a sample of a body fluid, to a sample of separated cells or to a sample from a tissue or an organ or to a sample of wash/rinse fluid obtained from an outer or inner body surface. Examples of such body samples include but are not limited to blood, lymph, urine, gynecological fluids, biopsies, and smears. Body samples may be obtained from a patient by a variety of techniques including, for example, by scraping or swabbing an area or by using a needle to aspirate bodily fluids. The sample comprises polynucleotides, for example the sample comprises DNA or RNA from one or more than one HPV. Samples can be obtained by well known techniques and include, for example scrapes or biopsies from the urogenital tract, perianal regions, anal canal, the oral cavity, the upper aerodigestive tract and the epidermis. Such samples can be obtained by use of brushes, cotton) swabs, spatula, rinse/wash fluids, punch biopsy devices, puncture of cavities with needles or surgical instrumentation. For example, samples of blood, plasma, serum, urine, saliva, lacrimal fluid, stool are encompassed by the method of the present disclosure. Tissue or organ samples may be obtained from any tissue or organ by, e.g., biopsy or other surgical procedures. Separated cells may be obtained from the body fluids or the tissues or organs by separating techniques such as filtration, centrifugation or cell sorting. The samples may be obtained for example from plasma, whole blood or peripheral blood mononuclear cell (PBMC) or a combination thereof. It is to be understood that the sample may be further processed in order to carry out the method of the present disclosure. Particularly, polynucleotides such as DNA or RNA, might be extracted and/or purified from the obtained sample by methods and means known in the art (e.g., see Examples). Thus, the term sample also may relate to polynucleotides, such as for example DNA or RNA, purified and/or extracted from any sample as mentioned above. Methods for collecting various body samples are well known in the art. The terms “subject” and “patient”, which are used interchangeably herein, refer to a human. More specifically the term “subject” and “patient” refers to a human with a diagnosed condition, disease, disorder or syndrome.

By the term “condition”, “disease”, “syndrome” or “disorder” it refers to for example, an autoimmune disorder such as for example. Systemic lupus erythematosus ISLE) or Multiple Sclerosis (MS), a chronic neurodegenerative disease such as for example, Alzheimer's disease, Parkinson's disease or Amyotrophic lateral sclerosis (ALS), or a neurodevelopmental disorder such as for example. Autism or autism spectrum disorders such as Asperger syndrome. The condition, disease, syndrome or disorder may further be Chronic Fatigue Syndrome, Fibromyalgia, Idopathic Lymphocytopenia, Depression, Seizures or Epilepsy. The condition, disease, syndrome or disorder may further be a metabolic disorder, such as for example, Neimann Pick Disease. The condition, disease, syndrome or disorder may further be lymphoma or leukemia.

Methods of detecting and/or typing of HPV types or variants is known in the art. As described herein methods are used to determine the HPV viral load in a sample by detecting nucleic acids associated with HPV, which in turn provides a basis for medical diagnosis, treatment, monitoring or evaluation of the condition.

Typing may be performed by using for example conventional DNA sequencing techniques. Dideoxy (Sanger) sequencing (Sanger et al., 1977 Proc Natl Acad Sci USA. 1977 December; 74(12): 5463-5467), Pyrosequencing (Ronaghi et al., Genome Res, 2001. 11: 3-11) and Mass Spectrometry DNA sequencing (Berkenkamp et al., 1998 Science 281 (5374): 260-262 10 Jul. 1998) or any other DNA-sequencing technique applicable to this technique may be used. Usually an oligonucleotide primer may be designed, where the primer may be able to hybridize to the single stranded form of the sample close to the variable site or region of interest in question. Hereby, the primer may be extended over the variable site or region of interest during sequencing, and thus the nucleotide pattern of the variable site or region of interest is determined i.e., the sample strand may be typed.

Furthermore, HPV can be typed for example by cross-hybridization. HPVs may be grouped into types based on the similarity of their DNA sequence. Two HPVs are taxonomically classified as being of the same type if their DNAs cross-hybridize to greater than 50%, as measured by hybridization in solutions under moderately stringent hybridization conditions, which are defined as approximately 25° C. below the melting temperature of a perfectly base-paired double-stranded DNA (conveniently written as Tm-25° C.), followed by chromatography on hydroxyapatite to separate double-stranded DNA from single-stranded DNA (see Coggin, J. R., et al., Cancer Res. 39:545 (1979)). The melting temperature (Tm) of a perfectly base-paired double-stranded DNA can be accurately predicted using the following well-established formula:

Tₘ=16.6×log[Na⁺]+0.41×% G:C+81.5−0.72×(%)(v/v) formamide

The above formula provides a convenient means to set a reference point for determining non-stringent and stringent hybridization conditions for various DNAs in solutions having varying salt and formamide concentrations without the need for empirically measuring the Tm for each individual DNA in each hybridization condition. If less than 50% of the respective HPV DNAs are able to cross-hybridize in solutions under moderately stringent conditions to form fully or partially double-stranded structures, as measured and defined by the ability to bind to hydroxyapatite, then the HPV DNAs are not sufficiently related to be taxonomically classified as being of the same type. A cut-off of 50% cross-hybridization using this method is employed as the consensus criterion for the assignment of novel HPV types for nomenclature purposes. This method for measuring the degree of cross-hybridization between HPV DNAs has been historically adopted as the method to be used to determine whether two HPV DNAs represent different isolates of a common type or represent isolates of different types.

Furthermore, various hybridization techniques may be used for typing. For example the HPV genotype might be determined by Southern hybridization assay. For example amplifying the HPV gene from the sample may be used to perform a reverse blot hybridization of the amplified HPV DNA using Southern hybridization on a membrane attached with a probe that can be specifically attached to HPV types.

The presence of a specific HPV target nucleic acid in a sample may also be determined by using peptide-nucleic acid (PNA) probes. Sample cells are suspended in a solution; one or more than one HPV target nucleic acids are isolated from the sample cells; the target nucleic acids is contacted with at least one PNA probe that is substantially complementary to at least a portion of a nucleic acid, the detection of which is desired; and hybridization between the PNA probe and a target nucleic acid detects the HPV target.

Furthermore restriction analysis may be performed on amplified DNA from the sample to determine HPV types. For example the resulting product of HPV DNA amplification as described above may subsequently be analyzed by restriction mapping by for example, using four independent restriction reactions, such as Ddel (5′-C/TN AG-3′), Pstl (5′-CTGCA/G-3′X HaeIII (5′-GG/CC-3′) and Rsal (5′-GT/AC-3′). However, any other restriction endonucleases that recognize the same sequences (isoschizomers) may also be used. The restrictions products obtained may subsequently be analyzed in agarose or polyacrylamide gels pre-stained with a nucleic acid stain for DNA (EtBr, SYBR green, Gel star, etc) or using any other assay which have been proposed for DNA fragment size measurements and which is known in the art. HPV type identification may be performed by comparison of restriction fragment length polymorphism analysis patterns obtained for each restriction enzyme with the reference restriction fragment length polymorphism analysis patterns.

The current method may also include a step of treating, reducing viral load or preventing infection with one or more than one HPV in a subject. Various vaccines against HPV types are known in the art for example GARDASIL™ and CERVARIX™ may prevent infection with HPV. Furthermore, patients infected with HPV may he treated as is known in the art. For example U.S. Pat. No. 8,663,964, herein incorporated by reference, discloses that two RNases (Ranpimase and the RNAse disclosed in U.S. Pat. No. 5,728,805, herein incorporated by reference) have been discovered to be active against certain HPV infections. Ranpimase is known to be non-toxic and well-tolerated in humans, and the other RNase is believed to share these qualities. Therefore subjects infected with HPV may be treated using these RNases by administering them in therapeutically effective amounts.

Furthermore anti-viral agents or combinations may be used to treat an HPV infection. An anti-viral agent may be an anti-viral compound or pharmaceutical composition including an anti-viral compound or combination. Examples of anti-viral agents that may be used to manage or treat HPV are for example Cidofovir (Vistideg), Brincifovir (cmx001), Lopinavir, Bleomycin: Dihydroartemisinin DiindolyImethane, synthetic long peptides or HIV protease inhibitors (see for example Van Valckenborgh et al. Clin Infect Dis. (2001)32 (3), Amine et al. PLoS ONE. 2009; 4(3): e501.8., Snoeck et al. Verh K Acad Geneeskd Belo. 2001; 63(2):93-120, De Schutter et al. Mol Cancer. 2013; 12: 158, U.S. Pat. No. 7,094,772, Batman et al, Antiviral Therapy 2011; 16:515-525, Hampson et al. September 2014, Vol. 12, No. 9, Pages 1021-1023, Adalatkhah et al. Dermatology Online Journal, 13(3), 2007, James et al. 1993 Volume 28, Issue 6, Pages 933-937, Disbrow et al. Cancer Res. 2005 Dec. 1; 65(23):10854-61, U.S. Pat. No. 7,989,491, EP 1446121, Kenter et al. N Engl J Med. 2009 Nov. 5; 361(19):1838-47, Kenter et al. Clin Cancer Res Jan. 1, 2008 14; 169, which are herewith incorporated by reference.

### EXAMPLES

### Samples

Samples were for example obtained from commercial bio-banks. Samples included plasma samples from patients that had been diagnosed with autism, systemic lupus erythematosus (SLE), Fibromyalgia and Multiple Sclerosis (MS). Furthermore, peripheral blood mononuclear cell (PBMC) samples from a patient with systemic lupus erythematosus (SLE) and with Alzheimer's disease (AD) were obtained. In addition, plasma and blood samples were obtained from patient with systemic lupus erythematosus (SLE) (see Table 1).

TABLE 1
Analyzed Samples

| Sample ID | Diagnosis | Sample Type | Sex | Age |
|---|---|---|---|---|
| 52 | Autism | Plasma | Male | 51 |
| 92 | Autism | Plasma | Male | 29 |
| 59 | SLE | PBMC | Female | 44 |
| 41 | SLE | Plasma | Female | 49 |
| 36 | SLE | Plasma | Female | 43 |
| 417 | Fibromyalgia | Plasma | Female | 72 |
| 117 | Fibromyalgia | Plasma | Female | 49 |
| 05 | AD | PBMC | Female | 79 |
| 69 | MS | Plasma | Male | 41 |
| 44 | SLE | Plasma | Male | 43 |
| 44 | SLE | Blood | Male | 43 |

### DNA Extraction

DNA was extracted with the Quick-gDNA™ Blood MidiPrep kit (Zymo Research) or Bioline Isolate II Blood DNA Kit from blood plasma, whole blood or from peripheral blood mononuclear cell (PBMC) according to manufacturer's recommendations and instructions. These results are summarized in Table 2.

TABLE 2
DNA extracted from samples

| | | Assay Tube | | Stock | Qubit Total |
|---|---|---|---|---|---|
| Sample ID | Sample Type | Conc. | | Conc. (ng) | DNA (ng) |
| (52) | Plasma | 46.7 | ng/ml | 9.34 | 149.44 |
| (92) | Plasma | 49.9 | ng/ml | 9.84 | 157.44 |
| (59) | PBMC | 1.17 | μg/ml | 226 | 4294.0 |
| (41) | Plasma | 11.2 | ng/ml | 2.24 | 35.84 |
| (36) | Plasma | 14.4 | ng/ml | 2.88 | 46.08 |
| (417) | Plasma | 42.5 | ng/ml | 8.5 | 136 |
| (117) | Plasma | 53 | ng/ml | 10.6 | 169.6 |
| (05) | PBMC | 1.22 | μg/ml | 244 | 4636.0 |
| (69) | Plasma | 7.88 | ng/ml | 1.58 | 30.02 |
| (44) | Plasma | 53 | ng/ml | 10.6 | 201.4 |
| (44) | Whole Blood | 65.6 | ng/ml | 13.1 | 248.9 |

### Detection of HPV DNA by PCR

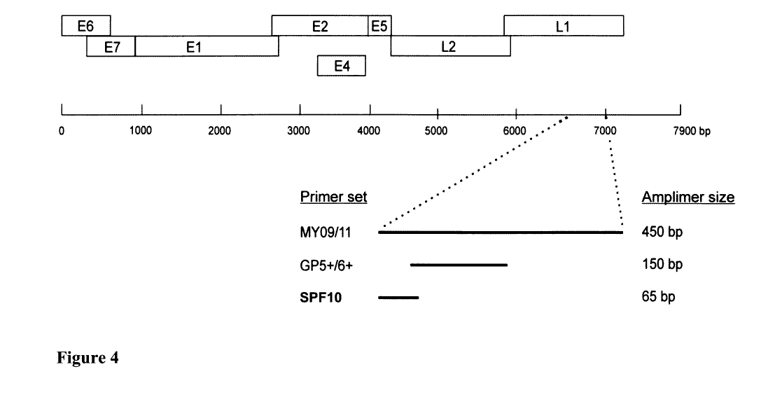

HPV DNA was detected in samples by PCR using either primers GP5+/6+ or PGMY 09/11 L1. Universal primer GP5+/6+ amplify the L1 region of the HPV viral genome. The pair GP5+/GP6+ flanks a sequence of approximately 150 pb (see FIG. 4). The pair GP5+/GP6+ consists of a fixed sequence of nucleotide for each oligonucleotide primer (See: de Roda Husman et al. J Gen Virol. 1995 April; 76 (Pt 4):1057-62).

PGMY 09/11 is a set of oligonucleotide pools, PGMY09 and PGMY11, based on the same primer binding regions used for MY09/11 (see FIG. 4) Rather than using the degenerate primer method, the PGMY09/11 primer system uses 5 upstream oligonucleotides comprising the PGMY11 primer pool and a set of 13 downstream oligonucleotides comprising the PGMY09 primer pool. The oligonucleotides were designed based on sequence homology of viruses in each of the two primer binding regions (See: Gravitt et al. J. Clin. Microbiol. January 2000, vol. 38, no. 1357-361).

GP5+/GP6+ PCR was performed in a final reaction volume of 25 μl containing 8.5 μl of template DNA, 12.5 μl SYBR® Green Supermix and 2 μl of each of the forward and reverse primers. The PCR conditions were as follows: activation for 10 min at 95° C. was followed by 60 cycles of 30 s at 95° C., 20 s at 48° C., and 30 s at 72° C.

PGMY09/11 was performed in a final reaction volume of 50 μl containing 8 of template DNA, 25 μl SYBR® Green Supermix and 17 μl of primers PGMY09/11. The PCR conditions were as follows: activation for 10 min at 94° C. was followed by 60 cycles of 30 s at 95° C., 20 s at 57° C., d 30 s at 68° C.

TABLE 3
Detection of HPV DNA in Samples by PCR

| Sample | | Sample | | | GP5+/ | PGMY09/ |
|---|---|---|---|---|---|---|
| ID | Diagnosis | Type | Sex | Age | 6+ Ct | 11 Ct |
| (92) | Autism | Plasma | Male | 29 | 28.0 | 29.1 |
| (47) | Multiple | Plasma | Female | 35 | 27.1 | 29.5 |
| | Sclerosis | | | | | |
| (417) | Fibromyalgia | Plasma | Female | 72 | 30.1 | 30.5 |
| (41) | SLE | Plasma | Female | 49 | 32.8 | 29.9 |
| (44) | SLE | Plasma | Male | 43 | 23.2 | 27.2 |
| (44) | SLE | Blood | Male | 43 | — | 24.5 |
| (Pos.) | Positive Ctrl | HPV pos. | — | — | — | 26.8 |

As can be seen in Table 3, two patients diagnosed with SLE tested positive for HPV in the plasma and blood samples when using primers PGMY09/11. and positive in the plasma samples when using primers GP5+/6+. Furthermore, the plasma samples of the patient with autism, Multiple Sclerosis (MS) and Fibromyalgia also tested positive for HPV.

### Quantitative Analysis of HPV DNA by PCR

Quantitative PCR was performed to assess the number of HPV DNA copies in the samples by using universal primers human papillomaviras (HPV). HPV was detected in the extracted DNA of the samples by amplifying viral DNA by PCR with universal primers GP5+/6+ and SPF10 (see FIG. 4). These primers are effective for amplifying wide spectrum of HPV genotypes. Quantitative analysis of HPV DNA and viral load determination was based on the studies as described by Forslund et al., J of General Virology 1999; Gravitt et al., J of Clinical Virology 2000; Smits et al., J of Clinical Microbiology 2005.

Sample Analysis with Universal Primer GP5+/6+

Universal primer GP5+/6+ amplify the L1 region of the HPV viral genome. The pair GP5+/GP6+ flanks a sequence of approximately 150 pb (see FIG. 4). The pair GP5+/GP6+ consists of a fixed sequence of nucleotide for each oligonucleotide primer (See: de Roda Husman et al. J Gen Virol, 1995 April; 76 (Pt 4):1057-62).

GP5+/GP6+ PCR was performed in a final reaction volume of 10 μl containing either 1 ng or 10 ng of isolated DNA, 5 μl SYBR® Green master mixes and 0.25 μl of each of the forward and reverse primers.

The PCR conditions were as follows: activation for 10 min at 95° C. was followed by 50 cycles of 30 s at 95° C., 20 s 50° C. and 30 s at 68° C., with a final extension of 7 min at 72° C. Each experiment was performed with separate positive and several negative PCR controls.

TABLE 4
Quantitative PCR of samples with GP5+/6+ primer pair

| | | | | | Qty | Qty |
|---|---|---|---|---|---|---|
| Sample ID | | Detector | Ct | Quantity | Mean | StdDev |
| 52 | (10 ng) | GP5+/6+ | Undetermined | 0 | | |
| 52 | (10 ng) | GP5+/6+ | Undetermined | 0 | | |
| 52 | (10 ng) | GP5+/6+ | Undetermined | 0 | | |
| 92 | (10 ng) | GP5+/6+ | Undetermined | 0 | | |
| 92 | (10 ng) | GP5+/6+ | Undetermined | 0 | | |
| 92 | (10 ng) | GP5+/6+ | Undetermined | 0 | | |
| 59 | (10 ng) | GP5+/6+ | 37.13774 | 50.33137 | 54.22361 | 5.504462 |
| 59 | (10 ng) | GP5+/6+ | 36.890755 | 58.11585 | 54.22361 | 5.504462 |
| 41 | (1 ng) | GP5+/6+ | 40.9497 | 5.468925 | 6.975 | 2.129912 |
| 41 | (1 ng) | GP5+/6+ | 40.19616 | 8.481075 | 6.975 | 2.129912 |
| 36 | (1 ng) | GP5+/6+ | Undetermined | 0 | | |
| 36 | (1 ng) | GP5+/6+ | Undetermined | 0 | | |
| 36 | (1 ng) | GP5+/6+ | Undetermined | 0 | | |
| 417 | (10 ng) | GP5+/6+ | Undetermined | 0 | | |
| 417 | (10 ng) | GP5+/6+ | Undetermined | 0 | | |
| 417 | (10 ng) | GP5+/6+ | Undetermined | 0 | | |
| 117 | (10 ng) | GP5+/6+ | Undetermined | 0 | | |
| 117 | (10 ng) | GP5+/6+ | Undetermined | 0 | | |
| 117 | (10 ng) | GP5+/6+ | Undetermined | 0 | | |
| 05 | (10 ng) | GP5+/6+ | 38.70348 | 20.22589 | 23.74355 | 4.974721 |
| 05 | (10 ng) | GP5+/6+ | 38.19082 | 27.26121 | 23.74355 | 4.974721 |
| 69 | (1 ng) | GP5+/6+ | 38.601517 | 21.46304 | 19.84983 | |
| 69 | (1 ng) | GP5+/6+ | 38.88129 | 18.23662 | 19.84983 | |

| | | | | |
|---|---|---|---|---|
| NTC + 10 ng hgDNA | GP5+/6+ | Undetermined | 0 | |
| NTC + 10 ng hgDNA | GP5+/6+ | Undetermined | 0 | 2.281423 |
| NTC + 10 ng hgDNA | GP5+/6+ | Undetermined | 0 | 2.281423 |
| 44 Plasma (10 ng) | GP5+/6+ | Undetermined | 0 | |
| 44 Plasma (10 ng) | GP5+/6+ | Undetermined | 0 | |
| 44 Plasma (10 ng) | GP5+/6+ | Undetermined | 0 | |
| 44 Whole Blood (10 ng) | GP5+/6+ | Undetermined | 0 | |
| 44 Whole Blood (10 ng) | GP5+/6+ | 40.8544 | 5.780965 | |

### Calculation of Viral Load

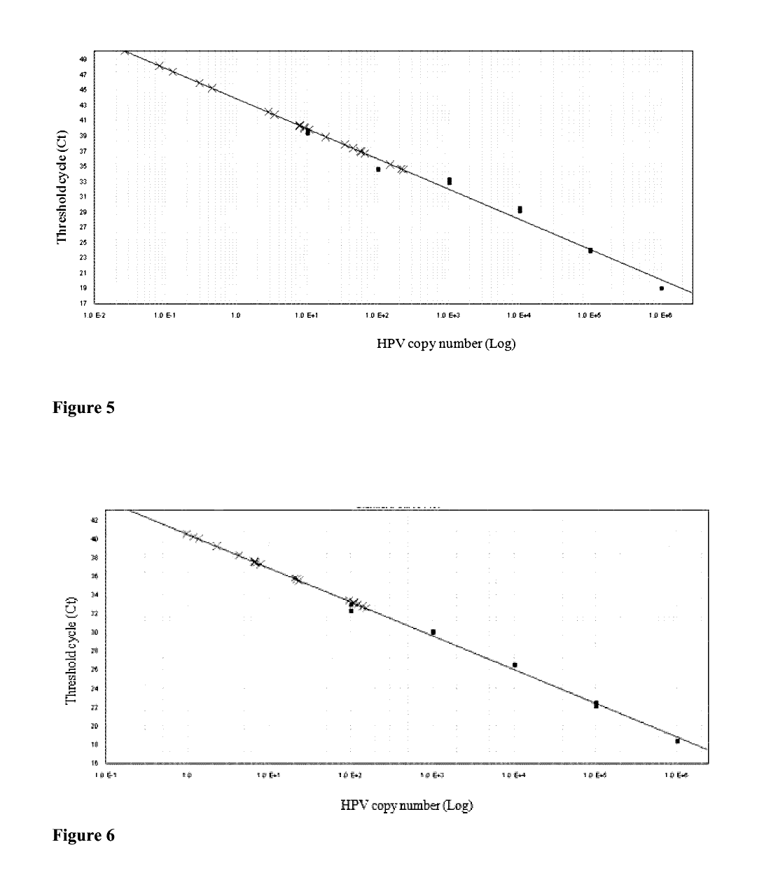

Standard calibration curves were generated automatically by plotting Ct values against the logarithm of the copy numbers of 10-fold serially diluted plasmid DNA for each primer pair used. Control plasmid was used to prepare a dilution series containing a known number of HVP 16 genome copies per reaction as well as 10 ng of commercial source human gDNA. All qPCR reactions were be set up with SYBR-Green powermix and the appropriate primer sets (as described above). HPV copy numbers in each DNA sample were calculated from the standard curves of HPV established from purified HPV plasmid DNA, respectively. The amount of HPV plasmid DNA (in nanograms) in each dilution was converted to HPV copy numbers from the known m.w. of the plasmids. SDS 2.4 software with Absolute Quantification mode was used to evaluate HPV copy numbers. Viral loads in each specimen were expressed as the number of HPV copies per reaction or copies per sample (see FIG. 5).

TABLE 5
Standard for GP5+/6+ primer pair

| Sample Name | Detector | Ct | # copies/rxn |
|---|---|---|---|
| Plasmid (1) + 10 ng hgDNA | GP5+/6+ | 39.130337 | 10 |
| Plasmid (1) + 10 ng hgDNA | GP5+/6+ | 39.096985 | 10 |
| Plasmid (2) + 10 ng hgDNA | GP5+/6+ | 35.59495 | 100 |
| Plasmid (2) + 10 ng hgDNA | GP5+/6+ | 35.360825 | 100 |
| Plasmid (3) + 10 ng hgDNA | GP5+/6+ | 33.23427 | 1000 |
| Plasmid (3) + 10 ng hgDNA | GP5+/6+ | 33.18077 | 1000 |
| Plasmid (4) + 10 ng hgDNA | GP5+/6+ | 29.218437 | 10000 |
| Plasmid (4) + 10 ng hgDNA | GP5+/6+ | 29.260967 | 10000 |
| Plasmid (5) + 10 ng hgDNA | GP5+/6+ | 24.3094 | 100000 |
| Plasmid (5) + 10 ng hgDNA | GP5+/6+ | 23.740639 | 100000 |
| Plasmid (6) + 10 ng hgDNA | GP5+/6+ | 19.040962 | 1000000 |
| Plasmid (6) + 10 ng hgDNA | GP5+/6+ | 19.152908 | 1000000 |
| −3.954573 | cycles/log decade | | |
| | 43.86779 | | |
| | 0.982312 | | |

### Viral Load GP5+/6+ Primer

HPV DNA was detected in 4 of 11 samples using the GP5+/6+ primers (see Table 8). Two of these samples were from patients with SLE and one sample each was from a patient with AD or MS. The HPV viral load ranged from 25 to 23,188 HPV copies/sample (7 to 54 copies/rxt) for detection with primer GP5+/6+. The viral load in patients with SLE that tested positive for HPV ranged from about 25 to 23188 copies/sample or 7 to 54 copies/reactions when the GP5+/6+ primers were used. The viral load for the patient with AD was 11126 copies/sample (24 copies/rxt) and for the patient with MS the viral load was 57 copies/sample (19 copies/rxt) for HPV detection with primer GP5+/6+ (see Table 8).

Sample Analysis with Universal Primer SPF10

The universal primer SPF10 target a conserved 65 bp region of the HPV L1 gene for broad-spectrum amplification (see FIG. 4). (Meter et al., 1998. Am. J. Pathol. 153:1731-1739).

SPF10 PCR was performed in a final reaction volume of 10 μl containing either 1 ng or 10 ng of isolated DNA, 5 μl SYBR® Green master mixes and 0.5 μl of the SPF10 primer pool.

The PCR conditions were as follows: activation for 10 min at 95° C. was followed by 50 cycles of 30 s at 95° C., 20 s at 50° C., and 30 s at 68° C., with a final extension of 7 min at 72° C. Each experiment was performed with separate positive and several negative PCR controls.

TABLE 6
Quantitative PCR of samples with SPF10 primer pair

| | | | | | Qty | Qty |
|---|---|---|---|---|---|---|
| Sample Name | | Detector | Ct | Quantity | Mean | StdDev |
| 52 | (10 ng) | SP | Undetermined | 0 | | |
| 52 | (10 ng) | SP | Undetermined | 0 | | |
| 52 | (10 ng) | SP | Undetermined | 0 | | |
| 92 | (10 ng) | SP | 40.10372 | 1.218084 | 1.10291 | 0.162881 |
| 92 | (10 ng) | SP | 40.431667 | 0.987736 | 1.10291 | 0.162881 |
| 92 | (10 ng) | SP | Undetermined | 0 | 1.10291 | 0.162881 |
| 59 | (10 ng) | SP | 32.873707 | 123.7957 | 126.0523 | 29.82208 |
| 59 | (10 ng) | SP | 32.50258 | 156.9386 | 126.0523 | 29.82208 |
| 59 | (10 ng) | SP | 33.248516 | 97.42263 | 126.0523 | 29.82208 |
| 41 | (1 ng) | SP | 35.658897 | 20.87113 | 22.20699 | 1.470992 |
| 41 | (1 ng) | SP | 35.45454 | 23.78345 | 22.20699 | 1.470992 |
| 41 | (1 ng) | SP | 35.57888 | 21.96637 | 22.20699 | 1.470992 |
| 36 | (1 ng) | SP | 39.89098 | 1.395513 | 2.327004 | 1.779905 |
| 36 | (1 ng) | SP | 38.10178 | 4.379351 | 2.327004 | 1.779905 |
| 36 | (1 ng) | SP | 40.11913 | 1.206147 | 2.327004 | 1.779905 |
| 417 | (10 ng) | SP | Undetermined | 0 | | |
| 417 | (10 ng) | SP | Undetermined | 0 | | |
| 417 | (10 ng) | SP | Undetermined | 0 | | |
| 117 | (10 ng) | SP | Undetermined | 0 | | |
| 117 | (10 ng) | SP | Undetermined | 0 | | |
| 117 | (10 ng) | SP | Undetermined | 0 | | |
| 05 | (10 ng) | SP | 33.014706 | 113.1266 | 121.7315 | 18.51908 |
| 05 | (10 ng) | SP | 32.648228 | 142.9875 | 121.7315 | 18.51908 |
| 05 | (10 ng) | SP | 33.07169 | 109.0803 | 121.7315 | 18.51908 |
| 69 | (1 ng) | SP | Undetermined | 0 | | |
| 69 | (1 ng) | SP | Undetermined | 0 | | |
| 69 | (1 ng) | SP | 37.344147 | 7.107653 | | |

| | | | | | |
|---|---|---|---|---|---|
| NTC + 10 ng hgDNA | SP | 15.718742 | 7159249 | | |
| NTC + 10 ng hgDNA | SP | Undetermined | 0 | | |
| NTC + 10 ng hgDNA | SP | Undetermined | 0 | | |
| 44 Plasma (10 ng) | SP | 39.108135 | 2.301701 | | |
| 44 Plasma (10 ng) | SP | Undetermined | 0 | | |
| 44 Plasma (10 ng) | SP | Undetermined | 0 | | |
| 44 Whole Blood (10 ng) | SP | 37.14999 | 8.046805 | 7.154048 | 0.781418 |
| 44 Whole Blood (10 ng) | SP | 37.408543 | 6.821033 | 7.154048 | 0.781418 |
| 44 Whole Blood (10 ng) | SP | 37.46143 | 6.594304 | 7.154048 | 0.781418 |

### Calculation of Viral Load

Standard calibration curves by using SPF10 primer were generated as described above (see FIG. 6).

TABLE 7
Standard for SPF10 primer pair

| Sample Name | Detector | Ct | # copies/rxn |
|---|---|---|---|
| Plasmid (2) + 10 ng hgDNA | SP | 32.416283 | 100 |
| Plasmid (2) + 10 ng hgDNA | SP | 32.321033 | 100 |
| Plasmid (2) + 10 ng hgDNA | SP | 32.944916 | 100 |
| Plasmid (3) + 10 ng hgDNA | SP | 30.196041 | 1000 |
| Plasmid (3) + 10 ng hgDNA | SP | 30.126915 | 1000 |
| Plasmid (3) + 10 ng hgDNA | SP | 30.049389 | 1000 |
| Plasmid (4) + 10 ng hgDNA | SP | 26.563625 | 10000 |
| Plasmid (4) + 10 ng hgDNA | SP | 26.528362 | 10000 |
| Plasmid (4) + 10 ng hgDNA | SP | 26.608019 | 10000 |
| Plasmid (5) + 10 ng hgDNA | SP | 22.305265 | 100000 |
| Plasmid (5) + 10 ng hgDNA | SP | 22.506006 | 100000 |
| Plasmid (5) + 10 ng hgDNA | SP | 22.10236 | 100000 |
| Plasmid (6) + 10 ng hgDNA | SP | 18.489363 | 1000000 |
| Plasmid (6) + 10 ng hgDNA | SP | 18.494143 | 1000000 |
| Plasmid (6) + 10 ng hgDNA | SP | 18.39286 | 1000000 |
| −3.602348 | cycles/log decade | | |
| | 40.41236 | | |
| | 0.990541 | | |

### Viral Load SPF10 Primer

When using SPF10 primer to detect HPV DNA in samples, patients with Autism, systemic lupus erythematosus (SLE), Alzheimer's disease (AD) and multiple sclerosis (MS) tested positive. HPV DNA was detected in 7 samples of 11 samples using the SPF 10 primer see Table 8). Three of the samples were from patients with SLE, one sample was from a patient with autism, one sample was from a patient with AD and one sample was from a patient with MS. Overall the results showed that in all three patients with SLE, HPV DNA was detected in the samples. Furthermore, patients diagnosed with autism, AD and MS were also positive for HPV DNA. The highest viral loads were detected in PBMC samples from one patient with SLE (ID# 59) and one patient with AD (ID #05).

The HPV viral load ranged from 9 to 56559 HPV copies/sample (<1 to 126 HPV copies/rxt) for detection with primer SPF10. In the four patients with SLE the viral load ranged from 9 to 54104 HPV copies/sample (<1 to 126 HPV copies/rxt). The viral load for the patient with autism was around 16 copies/sample. The viral load for the patient with AD was 56559 copies/sample (122 copies/rxt) and for the patient with MS the viral load was 21 copies/sample (7 copies/rxt). For HPV detection with primer SPF10 (see Table 8).

### Determination of HPV Types by DNA Sequencing

The HPV types detected in the samples were determined by DNA sequencing, as generally known in the art. See for example Lee et al. Infectious Agents and Cancer 2007, 2:11 or Arroyo et al. J Clin Virol. 2013 October; 58(2):437-42.

TABLE 8
HPV DNA detection in blood from patients using L1 primers GP5+/6+ or SPF10

| | | | | | HPV | HPV | HPV | HPV | HPV | HPV | |
|---|---|---|---|---|---|---|---|---|---|---|---|
| Sample | | Sample | | | copies/rx * | copies/sample | copies/μl | copies/rx * | copies/sample | copies/μl | HPV |
| ID | Diagnosis | Type | Sex | Age | GP5+/6+ | GP5+/6+ | GP5+/6+ | SPF10 | SPF10 | SPF10 | type |
| 52 | Autism | Plasma | Male | 51 | 0 | 0 | 0 | 0 | 0 | 0 | |
| 92 | Autism | Plasma | Male | 29 | 0 | 0 | 0 | 1 | 16 | 1 | |
| 59 | SLE | PBMC | Female | 44 | 54 | 23188 | 5 | 126 | 54104 | 13 | 16 |
| 41 | SLE | Plasma | Female | 49 | 7 | 25 | 1 | 22 | 79 | 2 | 16 |
| 36 | SLE | Plasma | Female | 43 | 0 | 0 | 0 | 0 | 9 | 0 | |
| 417 | Fibromyalgia | Plasma | Female | 72 | 0 | 0 | 0 | 0 | 0 | 0 | |
| 117 | Fibromyalgia | Plasma | Female | 49 | 0 | 0 | 0 | 0 | 0 | 0 | |
| 05 | AD | PBMC | Female | 79 | 24 | 11126 | 2 | 122 | 56559 | 12 | 16 |
| 69 | MS | Plasma | Male | 41 | 19 | 57 | 2 | 7 | 21 | 1 | 16 |
| 44 | SLE | Plasma | Male | 43 | 0 | 0 | 0 | 2 | 0 | 0 | 16 |
| 44 | SLE | Blood | Male | 43 | 0 | 0 | 0 | 7 | 21 | 1 | 16 |

* the reaction volume is 10 μl

TABLE 9
HPV 16 variants detected

| | Sequence | | | Variants |
|---|---|---|---|---|
| SampleID | Length | Reference (HPV 16) | PercentID | (with respect to sample) |
| 59 | 106 | gi\|310698439\|ref\|NC_001526.2\| | 99.07 | −>T at position 102 |
| 41 | 110 | gi\|310698439\|ref\|NC_001526.2\| | 98.18 | G > T at position 12; |
| | | | | A > T at position 15 |
| 05 | 88 | gi\|310698439\|ref\|NC_001526.2\| | 100 | None |
| 69 | 83 | gi\|310698439\|ref\|NC_001526.2\| | 98.81 | −>T at position 79 |
| 44(WB) | 103 | gi\|310698439\|ref\|NC_001526.2\| | 96.12 | G > T at position 9; |
| | | | | A > T at position 12; |
| | | | | C > A at position 19; |
| | | | | G > A at position 32 |
| Plasmid(1) | 106 | gi\|310698439\|ref\|NC_001526.2\| | 99.07 | −>T at position 102 |
| Plasmid(2) | 100 | gi\|310698439\|ref\|NC_001526.2\| | 99.01 | −>T at position 96 |
| Plasmid(3) | 115 | gi\|310698439\|ref\|NC_001526.2\| | 99.14 | −>T at position 111 |

All citations are hereby incorporated by reference.

The present invention has been described with regard to one or more embodiments. However, it will be apparent to persons skilled in the art that a number of variations and modifications can be made without departing from the scope of the invention as defined in the claims.

1. A method of diagnosing, treating, quantifying, monitoring or evaluating a condition in a subject comprising determining the presence or quantity of one or more than one human papilloma virus (HPV) in a sample from the subject, wherein the presence or quantity of the one or more than one HPV is indicative of the presence of the condition in the subject or indicative of a predisposition to the condition when compared to a control, the condition comprising an autoimmune disorder, a chronic neurodegenerative disease, a neurodevelopmental disorder, neoplasm, blood cancers such as lymphoma or leukemia, Chronic Fatigue Syndrome or Fibromyalgia.

2. The method of claim 1, wherein the quantity comprises viral load of one or more than one human papilloma virus in the sample.

3. The method of claim 2 wherein determining the viral load comprises:
a) generating a standard curve with more than one known concentration of an HPV nucleic acid; and
b) calculating the viral load of the sample based on the standard curve.

4. The method of claim 2, wherein the viral load is determined by quantifying the genome numbers of the one or more than one human papilloma virus (HPV).

5. The method of claim 4, wherein the HPV genome numbers in the sample is greater than the genome numbers in a sample of the control.

6. The method of claim 4, wherein the genome numbers is determined by quantitative polymerase chain reaction (PCR).

7. The method of claim 4, wherein the genome number is greater than 2 copies per μl as measured by HPV-DNA quantitative PCR using universal primer GP5+/6+.

8. The method of claim 4, wherein the genome number is greater than 4 copies per μl as measured by HPV-DNA quantitative PCR using universal primer SPF10.

9. The method of claim 1, wherein the viral load is above a threshold amount;
the threshold amount being indicative of the presence of the condition in the subject or an increased likelihood of the subject developing the condition when compared to a control subject.

10. The method of claim 9, wherein the threshold amount is a copy number greater than 1 copies per μl as measured by HPV-DNA quantitative PCR using universal primer SPF10 and/or greater than 1 copies per μl measured by HPV-DNA quantitative PCR using universal primer Gp5+/6+.

11. The method of claim 1, wherein the autoimmune disorder is Systemic lupus erythematosus (SLE) or Multiple Sclerosis (MS).

12. The method of claim 1, wherein the chronic neurodegenerative disease is Alzheimer's disease, Parkinson's disease or Amyotrophic lateral sclerosis (ALS).

13. The method of claim 1, wherein the neurodevelopmental disorder is Autism or autism spectrum disorders such as Asperger syndrome.

14. The method of claim 1, wherein the sample is blood, plasma or peripheral blood mononuclear cell (PBMC) or other blood compartment or fluid such as cerebrospinal fluid or saliva.

15. The method of claim 1, further comprising treating the subject for one or more than one HPV infection or vaccinating the subject against one or more than one HPV.

16. The method of claim 1, wherein the one or more than one HPV comprises one or more than one HPV beta, one or more than one HPV gamma, one or more than one HPV alpha or a combination thereof.

17. The method of claim 1, wherein the one or more than one HPV comprises one or more than one high risk HPV type, one or more than one low risk HPV type, or a combination thereof.

18. The method of claim 17, wherein one or more than one high risk type HPV comprises one or more than one HPV16, HPV18, HPV31, HPV33, HPV 35, HPV 39, HPV 45, HPV 51, HPV 52, HPV 53, HPV 56, HPV 58, HPV 59, HPV 66, HPV 67, HPV 68, HPV 82, HPV 85 or a combination thereof.

19. The method of claim 17, wherein one or more than one low risk type HPV comprises one or more than one HPV2, HPV3, HPV6, HPV11, HPV13, HPV32, HPV40, HPV42, HPV43, HPV44, HPV57 or a combination thereof.

20. The method of claim 1, wherein one or more than one HPV comprises HPV type 1, 2, 4, 6, 11, 16, 18, 26, 30, 31, 33, 34, 35, 39, 40, 41, 42, 43, 44, 45, 51, 52, 54, 55, 56, 57, 58, 59, 61, 62, 64, 67, 68, 69, 70 or a combination thereof.

21. A method of comparing the incidence of a HPV infection among more than one population of subjects with a condition, the method comprising
a) determining the viral load of one or more than one HPV in a representative number of subjects of each population;
b) calculating the percentage of subjects in each population having a viral load above a threshold; and
c) correlating the percentage of subjects in each population to the relative incidence of HPV infection, wherein the condition comprises an autoimmune disorder, a chronic neurodegenerative disease, a neurodevelopmental disorder, neoplasm, blood cancers such as lymphoma or leukemia, Chronic Fatigue Syndrome or Fibromyalgia.

22. A method for predicting the development of a condition in a human subject, comprising contacting a biological sample of the human subject with a diagnostic reagent that can detect one or more than one human papillomavirus (HPV) or an antigen thereof, or a humoral or cell-mediated response to HPV or an antigen thereof, in the biological sample;
wherein the presence of one or more than one HPV or an antigen thereof or a humoral or cell-mediated response to HPV or an antigen thereof is associated with an increased risk of developing the condition, wherein the condition comprises an autoimmune disorder, a chronic neurodegenerative disease, a neurodevelopmental disorder, neoplasm, blood cancers such as lymphoma or leukemia, Chronic Fatigue Syndrome or Fibromyalgia.

23. A method of treating a condition related to HPV comprising:
administering a therapeutically effective amount of an antiviral, anti-retroviral or combination of antiviral agents to a to a subject in need thereof, wherein the condition comprises an autoimmune disorder, a chronic neurodegenerative disease, a neurodevelopmental disorder, neoplasm, blood cancers such as lymphoma or leukemia, Chronic Fatigue Syndrome or Fibromyalgia.

24. (canceled)
